(19) United States
(12) Patent Application Publication
(10) Pub. No.: US 2025/0195078 A1
(43) Pub. Date: Jun. 19, 2025

(54) SYSTEM AND METHOD FOR THE DELIVERY OF AN EMBOLIZATION DEVICE

(71) Applicant: CLEARSTREAM TECHNOLOGIES LIMITED, Enniscorthy, County Wexford (IE)

(72) Inventors: Michael WHELAN, Enniscorthy, County Wexford (IE); Ciaran GILES, Kildare (IE); Fearghal O'CONNOR, Gorey, County Wexford (IE)

(21) Appl. No.: 18/852,190

(22) PCT Filed: Mar. 31, 2022
(86) PCT No.: PCT/EP2022/058573

§ 371 Date: Sep. 27, 2024

(51) Int. Cl. A61B 17/12

(52) U.S. Cl. CPC A61B 17/12145; A61B 2017/1205

(57) ABSTRACT

A system for the delivery of an embolization device comprising a delivery wire and an expandable structure, wherein the system comprises a loading device comprising a compartment configured to contain the expanded structure of the embolization device; a conduit configured to receive the delivery wire of the embolization device, wherein the conduit is coupled to the compartment; and a fluid inlet in fluid communication with the compartment.

### CROSS-REFERENCE TO RELATED APPLICATIONS

The present application is a U.S. National Phase of International Application No. PCT/EP2022/058573, entitled “SYSTEM AND METHOD FOR THE DELIVERY OF AN EMBOLIZATION DEVICE,” filed Apr. 22, 2022, which is incorporated herein by reference in its entirety.

### TECHNICAL FIELD

The present disclosure relates to systems and methods for the delivery of medical devices, and more particularly to systems and methods for the delivery of embolization devices into a vessel of a patient.

### BACKGROUND

Catheter embolization is a well-established procedure involving the occlusion of a blood vessel to prevent blood flow to an area of the patient's body. Such a procedure may be required to control or prevent bleeding, or to treat a tumour or aneurysms.

Occlusion devices commonly comprise an expandable structure, often with a cover or coating, so that the implant, in a folded configuration, fits into the lumen of a delivery catheter and, in an expanded configuration, occludes the patient's vessel.

The overall delivery procedure may be simplified by pre-packaging the occlusion device in a sheath, thereby removing the step of loading the occlusion device into the sheath. Upstream, it may also minimise the amount of packaging and the storage space required for the equipment. However, this means that the occlusion device may be stored in a folded configuration in the constrained lumen of a sheath for extended periods of time, which results in the deterioration of the device in terms of integrity and subsequent deployment.

Instead, the occlusion device may be provided, in the expanded configuration, in a separate packaging. The device is carefully removed from the packaging and immersed in a bowl of saline, before being loaded into a sheath and connected to a delivery catheter. This method does not completely remedy the issues associated with occlusion devices pre-packaged in a sheath. The increased number of manipulations complicates the delivery procedure and additional time is required to load the occlusion device. There is also an increased risk of contamination. As in most medical procedures, the equipment, including the occlusion device, must be sterile/sterilised, and several sterilisation cycles may be required which could affect the integrity of the occlusion device.

It is an object of this disclosure to mitigate problems such as those described above and to provide an improved alternative to existing products. In particular, a need exists for an improved system and method for the delivery of embolization and occlusion devices.

### SUMMARY

According to a first aspect of the disclosure, there is provided a system for the delivery of an embolization device comprising a delivery wire and an expandable structure. The system comprises a loading device comprising a compartment configured to contain the expanded structure of the embolization device; a conduit configured to receive the delivery wire of the embolization device, wherein the conduit is coupled to the compartment; and a fluid inlet in fluid communication with the compartment.

In an embodiment, the loading device comprises a distal seal configured to selectively allow passage of a sheath therethrough. Additionally or alternatively, the loading device comprises a distal valve configured to selectively allow passage of a sheath therethrough. The distal seal and/or valve of the loading device may be coupled to a Tuohy Borst valve or a Y-connector.

In an embodiment, the loading device comprises a proximal seal configured to selectively allow fluid flow therethrough. Additionally or alternatively, the loading device comprises a proximal valve configured to selectively allow fluid flow therethrough. In an embodiment, the loading device comprises a proximal seal configured to selectively allow passage of a sheath therethrough and into the conduit. Additionally or alternatively, the loading device comprises a proximal valve configured to selectively allow passage of a sheath therethrough and into the conduit. Hereinbelow, the term “seal” will be used to refer to a seal, a valve, or a seal and a valve. The proximal seal and the distal seal may be aligned along the longitudinal axis of the loading device.

In an embodiment, the loading device comprises a fluid outlet.

In an embodiment, the loading device is made, partly or completely, of a transparent or semi-transparent material. The loading device may comprise polycarbonate, nylon and/or polytetrafluoroethylene (PTFE).

In an embodiment, the system comprises a sheath. In an embodiment, the system comprises a delivery catheter. In an embodiment, the system comprises a connector for connecting a sheath to a delivery catheter. In an embodiment, the system comprises an embolization device.

In an embodiment, the system comprises a source of fluid. The source of fluid may be fluidly connectable to the fluid inlet of the loading device.

According to a second aspect of the disclosure, there is provided a method for delivering an embolization device comprising a delivery wire and an expandable structure, wherein the method comprises the step of providing a loading device as described herein above.

In an embodiment, the method comprises the step of connecting a source of fluid to the fluid inlet of the loading device.

In an embodiment, the method comprises the step of delivering fluid into the loading device. The step of delivering the fluid may comprise the step of positioning the loading device substantially vertically during the fluid delivery step, so that the fluid is delivered to the proximal conduit first before the fluid is delivered to the distal compartment.

In an embodiment, the method comprises the step of substantially immersing the embolization device with fluid or flushing the embolization device with fluid.

In an embodiment, the method comprises the loading device comprises a proximal seal or valve, and the method comprises the step of inserting a sheath through said proximal seal and over the embolization device.

In an embodiment, the method comprises the loading device comprises a distal seal or valve, and the method comprises the step of inserting the sheath through said distal seal or valve.

In an embodiment, the method comprises the step of connecting the sheath with a delivery catheter.

In an embodiment, the method comprises the step of advancing the embolization device through the sheath and into the delivery catheter.

These and other features of the concepts provided herein will become more apparent to those of skill in the art in view of the accompanying drawings and the following description, which describe particular embodiments of such concepts in greater detail.

### BRIEF DESCRIPTION OF THE DRAWINGS

FIG. 1A is a schematic representation of a loading device for use in an embolization device delivery system, in accordance with embodiments described herein;

FIG. 1B is a schematic representation of an embolization device delivery system comprising a loading device of FIG. 1A, with the embolization device in an expanded configuration, in accordance with embodiments described herein;

FIG. 1C is a schematic representation of the embolization device delivery system FIG. 1B, with the embolization device in a folded configuration, in accordance with embodiments described herein;

FIGS. 2A and 2B are schematic representations of an embolization device delivery system comprising a loading device, in accordance with embodiments described herein.

### DETAILED DESCRIPTION

The embodiments described herein are provided as exemplary and non-limiting embodiments of the present disclosure.

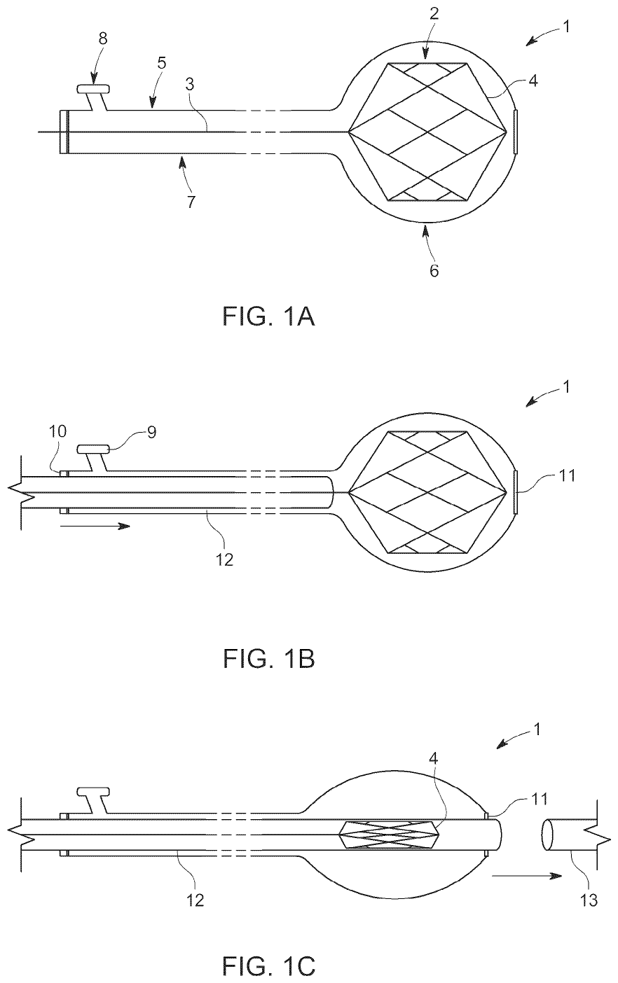

With reference to FIGS. 1A-1C, there is illustrated a system 1 for the delivery of an embolization device 2 comprising a delivery wire 3 and an expandable structure 4. The system 1 comprises a loading device 5 comprising a compartment 6 configured to contain the expanded structure 4 of the embolization device 2. The loading device 5 further comprises a conduit 7 configured to receive the delivery wire 3 of the embolization device 2. The conduit 7 is coupled to the compartment 6. The loading device further includes a fluid inlet 8 in fluid communication with the compartment 6.

The embolization device 2 (also referred to as an occlusion device, occlusion implant, embolization implant, or implant in the present disclosure) may be a micro-vascular plug (“MVP”).

The embolization device 2 comprises a delivery wire 3 and an expandable structure 4. The delivery wire 3 may be detachably coupled to the expandable structure 4.

The expandable structure 4 may be configured to expand from a first configuration in which the structure 4 is unexpanded so as to fit in a sheath and/or delivery catheter, to a second configuration in which the expandable structure 4 is expanded so as to occlude a patient's vessel. The expandable structure 4 may be configured to revert from the second configuration to the first configuration.

The expandable structure 4 may comprise an expandable scaffold. The scaffold may be expandable by material means (for example, it comprises or consists of a shape-memory material such as nitinol) and/or by mechanical means. The expandable structure 4 may comprise one or more expandable bodies, such as one or more expandable or inflatable balloons. The system 1 may comprise an inflation line to inflate the one or more inflatable balloons. The expandable structure 4 may comprise one or more biocompatible coatings or covers. In embodiments, the expandable structure 4 comprises a nitinol cage and a graft covering.

The loading device 5 is configured (i.e. shaped and dimensioned) to contain an embolization device 2 comprising a delivery wire 3 and an expandable structure 4. More particularly, the loading device 5 comprises a conduit 7 configured to receive the delivery wire 3 of the embolization device 2 and a compartment 6 configured to contain the expanded structure 4 of the embolization device 2.

The conduit 7 is coupled to the compartment 6 so as to contain the delivery wire 3 coupled to the expandable structure 4. The delivery wire 3 comprises a proximal end and a distal end, the proximal end being located opposite the distal end, and the expandable structure 4 may be coupled to the distal end of the delivery wire 3. Similarly, the conduit 7 comprises a proximal end and a distal end, the proximal end being located opposite the distal end. The compartment 6 may be coupled to the distal end of the conduit 7. The conduit 7 may be configured to allow fluid passage around the delivery wire 3.

The conduit 7 may be configured (i.e. shaped and dimensioned) to receive a sheath 12. The sheath 12 may be introduced from the proximal end of the conduit 7. The sheath 12 may be configured to slide over and receive the delivery wire 3 therethrough, and to be slidable from the proximal end towards the distal end of the conduit 7.

The loading device 5 may comprise a proximal seal 10 configured to selectively allow passage of the sheath 12 therethrough and into the conduit 7. The proximal seal 10 may be located at the proximal end of the conduit 7. In a closed configuration, the proximal seal 10 prevents fluid passage. The proximal seal 10 may selectively open to allow passage of the sheath 12. The proximal seal 10 may comprise a seal configured to surround the outer circumference of the sheath 12 so as to prevent fluid leakage.

The proximal seal 10 may allow passage of other components, such as the delivery wire 3. The proximal seal 10 may comprise an opening configured to surround and support the delivery wire 3. The opening may be centrally positioned in the proximal seal 10. The proximal seal 10 may comprise a seal configured to surround the outer circumference of the delivery wire 3 so as to prevent fluid leakage. The delivery wire 3 may be slidably received through the opening of the proximal seal 10. The opening may be selectively openable, for example to allow the insertion of a detachable delivery wire 3, i.e. a detachable delivery wire 3 may be inserted through the proximal seal 10, advanced to the expandable structure 4 located in the compartment 6 and coupled to the expandable structure 4.

The conduit 7 may be integrally made with the compartment 6, or coupled to the compartment 6. The conduit 7 may be made of the same material(s) as the compartment 6, or of different material(s). The conduit 7 may be made of a rigid or semi-rigid material so as to facilitate manipulation of the loading device 5. In particular, a rigid or semi-rigid material facilitates the insertion of the sheath 12 into the loading device 5 and also protects the delivery wire 3 from damage in transport and storage. In an embodiment, both the conduit 7 and the compartment 6 are made of a rigid or semi-rigid material so as to protect the connection between the delivery wire 3 and the expandable structure 4.

The compartment 6 is configured to contain the expandable structure 4 in its expanded configuration. When stored in the expanded configuration, the risk of deterioration of the device in terms of implant integrity and shape recovery is minimised. The compartment 6 may comprise or be made of a rigid or semi-rigid material so as to protect the integrity of the embolization device 2 from potential damage during transport and storage, for example from physical impact or pressure exerted onto the loading device 5.

The compartment 6 may be configured to hold and/or cradle the embolization device 2. For example, the shape of the compartment 6 may substantially follow the outer contours of the expanded structure 4. The dimensions of the compartment 6 (relative to the dimensions of the expanded structure 4) may be small enough to prevent excessive movement of the expanded structure 4 within the compartment 6, in particular during transport and manipulation prior to use. The dimensions of the compartment 6 (relative to the dimensions of the expanded structure 4) may be such that they allow passage of fluid between the inner surface of the compartment 6 and the expanded structure 4.

The compartment 6 and/or the conduit 7 may be made of the transparent or semi-transparent material. For example, the compartment 6 and/or the conduit 7 may be made or comprise polycarbonate, polytetrafluoroethylene (PTFE), nylon and the like. This enables the user to visualise and inspect the implant prior to and during the delivery process.

The loading device 5 may comprise a distal seal and/or valve 11 configured to selectively allow passage of a sheath therethrough. The distal seal and/or valve 11 may be coupled to the expandable structure 4. The distal seal and/or valve 11 may be coupled to the expandable structure 4 so as to be aligned with the longitudinal axis of the conduit 7.

The distal seal and/or valve 11 may be the same or different from the proximal seal 10 in structure and function. The distal seal and/or valve 11 may be or comprise a connector, such as a Tuohy Borst valve or a Y-connector.

The loading device 5 comprises a fluid inlet 8. The inlet 8 is in fluid communication with the inner space of the loading device 5, in particular with the inner space of the compartment 6 containing the expandable structure 4. The fluid inlet 8 may be positioned on the conduit 7 or on the compartment 6. In an embodiment, the fluid inlet 8 is positioned adjacent the proximal end of the conduit 7.

The fluid inlet 8 is configured to deliver fluid into the loading device 5, for example from a source of fluid. The source of fluid may be any suitable reservoir or a syringe, for example a pre-filled syringe.

The fluid may be a saline solution. Additionally or alternatively, the fluid may comprise one or more agents selected from a sterilising agent, an antibacterial agent, a lubricating agent, and a drug compound.

The delivery system 1 may comprise a source of fluid. The delivery system 1 may comprise the sheath 12, a delivery catheter 13 and a connector for connecting the sheath to the delivery catheter 13. The delivery system 1 may comprise the embolization device 2.

In an embodiment, the delivery system 1 may be provided as a kit comprising the loading device 5 and one or more of the embolization device 2, the sheath 12, the delivery catheter 13, a connector for connecting the sheath 12 to the delivery catheter 13, and a source of fluid. The delivery system 1 may for example be provided with the embolization device 2 contained within the loading device 5, or the loading device 5 may be provided separately. The loading device 5 may be provided with the expandable structure 4 contained therein, with a connectable delivery wire provided together or separately.

An exemplary method is described in which the delivery system 1 is provided with the embolization device 2 contained within the loading device 5, as illustrated for example in FIG. 1A.

The expandable structure 4 is contained in an expanded configuration in the compartment 6 of the loading device 5. The delivery wire 3 is coupled to and extends from the expandable structure 4 and is contained in the conduit 7 of the loading device 5. The delivery wire 3 extends through the central opening (not shown) of the proximal seal 10.

A source of fluid (not shown) is fluidly connected to the fluid inlet 8. Fluid is delivered, through fluid inlet 8, into the conduit 7 and the compartment 6, which are in fluid communication with each other. Therefore, the loading device 5 functions as a flushing device. The fluid contacts and flushes or immerses the embolization device 2. The embolization device 2 is flushed in the enclosed inner space of the loading device 5 so that all surfaces of the embolization device 2 are in contact with the fluid. This flushing method is effective in removing any air bubbles trapped in and around the embolization device 2, thereby minimising the risk of embolism in the patient.

The embolization device 2 can be flushed without being taken out of the loading device 5 and without the need for a separate saline bath. The loading device 5 therefore functions as a protective packaging for the embolization device 2. In addition, the number of manipulations is decreased, so that the delivery procedure is simpler, and the risk of contamination of or damage to the embolization device 2 is minimised.

The fluid may fill the loading device 5 in a single load, and remain in the loading device 5, which is sealed by means of the proximal seal 10 and the distal seal and/or valve 11. Alternatively, the loading device 5 may include a fluid outlet, or one of the proximal or distal seal 10, 11 may be open so as to allow fluid to flow through the loading device 5. The fluid outlet, the proximal seal 10 and/or the distal seal 11 may be or comprise an airlock seal, preventing air from re-entering the loading deliver 5.

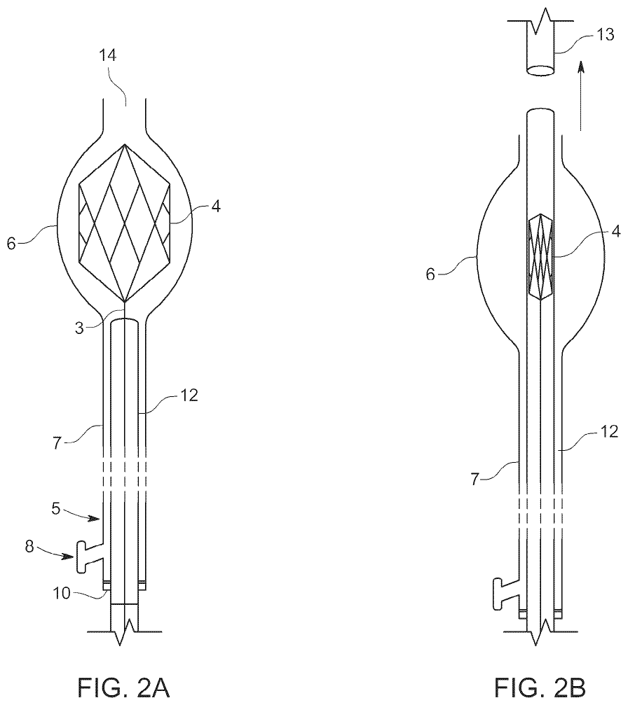

Alternatively or additionally, the loading device 5 is positioned substantially vertically as illustrated in FIGS. 2A and 2B. Fluid is delivered through fluid inlet 8, and fills the loading device 5 from its proximal (now bottom) end to its distal (now top) end. The distal end 14 of the loading device 5 is open so that excess fluid exits therethrough. This positioning allows air bubbles to be more effectively removed through the distal end 14 of the loading device 5.

The exiting fluid may be discarded or recirculated through the system 1. For example, an outlet line may be connected to the distal end 14 of the loading device 5 to remove excess fluid and air bubbles.

With reference to FIG. 1B, sheath 12 is inserted through the proximal end of the conduit 7 (FIG. 1A). This may be effected by means of a seal (for example a breakable seal), a valve and/or a connector, such as the proximal seal 10, coupled to the proximal end of the conduit 7.

The sheath 12 is advanced along the lumen of the conduit 7 and over the delivery wire 3. Upon reaching the compartment 6, the sheath 12 will contact the proximal end of the expanded structure 4 and progressively advance over the expandable structure 4 to fold the structure 4.

With reference to FIG. 1C, the expandable structure 4 is now in a folded configuration within the sheath 12, and the sheath 12 is ready to exit the loading device 5. This may be effected by means of a seal (for example a breakable seal), a valve and/or a connector, such as the distal seal and/or valve 11 (FIG. 1B), coupled to the distal end of the loading device 5.

The sheath 12 may be coupled to the delivery catheter 13, for example, by means of a connector (not shown), and the embolization device 2 may be loaded into the delivery catheter 13. The loading device 5 therefore serves the function of loading the embolization device 2 into the delivery catheter 13. The embolization device 2 is now ready for insertion into the patient.

The loading device 5 is an extracorporeal or external device in that it is not inserted into the patient and remains outside the body of the patient. Once the embolization device 2 is loaded in the delivery catheter, the loading device 5 is removed.

Thus, the present disclosure provides a system for the delivery of an embolization device into a patient. The delivery system protects the embolization device from damage, for example from physical impact, during storage, transport and use, and from contamination. The embolization device is contained in an expanded configuration so that to preserve the integrity of the implant and to prevent deterioration of the expansion mechanism. The delivery system allows the user to manipulate and flush the embolization device without taking the implant out of the loading device and without the need for a separate saline bath. Therefore, the delivery procedure is simpler, and the risk of contamination of or damage to the embolization device is minimised.

1. A system for the delivery of an embolization device comprising a delivery wire and an expandable structure, wherein the system comprises a sheath connectable to a delivery catheter, and a loading device comprising:
a compartment configured to contain the expanded structure of the embolization device;
a conduit configured to receive the delivery wire of the embolization device, wherein the conduit is coupled to the compartment; and
a fluid inlet in fluid communication with the compartment.

2. The system according to claim 1, wherein the loading device comprises a distal seal configured to selectively allow passage of the sheath therethrough.

3. The system according to claim 2, wherein the distal seal of the loading device is coupled to a Tuohy Borst valve or a Y-connector.

4. The system according to claim 1, wherein the loading device comprises a proximal seal configured to selectively allow fluid flow therethrough.

5. The system according to claim 1, wherein the loading device comprises a proximal seal configured to selectively allow passage of the sheath therethrough and into the conduit.

6. The system according to claim 4, wherein the proximal seal and the distal seal are aligned along the longitudinal axis of the loading device.

7. The system according to claim 1, wherein the loading device further comprises a fluid outlet.

8. The system according to claim 1, wherein the loading device is made, partly or completely, of a transparent or semi-transparent material.

9. The system according to claim 8, wherein the loading device comprises polycarbonate, nylon and/or polytetrafluoroethylene (PTFE).

10. The system according to claim 10, wherein the system further comprises a delivery catheter.

11. The system according to claim 1, wherein the system further comprises the embolization device.

12. The system according to claim 1, wherein the system further comprises a source of fluid, said source of fluid being fluidly coupled to the fluid inlet of the loading device.

13. A method for delivering an embolization device comprising a delivery wire and an expandable structure, wherein the method comprises the step of providing a loading device comprising:
a compartment configured to contain the expanded structure of the embolization device;
a conduit configured to receive the delivery wire of the embolization device, wherein the conduit is coupled to the compartment; and
a fluid inlet in fluid communication with the compartment.

14. The method according to claim 13, further comprising the step of connecting a source of fluid to the fluid inlet of the loading device.

15. The method according to claim 13, further comprising the step of delivering fluid into the loading device.

16. The method according to claim 15, further comprising the step of positioning the loading device substantially vertically during the fluid delivery step, so that the fluid is delivered to the proximal conduit first before the fluid is delivered to the distal compartment.

17. The method according to claim 13, further comprising the step of substantially immersing the embolization device with fluid or flushing the embolization device with fluid.

18. The method according to claim 13, wherein the loading device comprises a proximal seal, and wherein the method further comprises the step of inserting a sheath through said proximal seal and over the embolization device.

19. The method according to claim 18, wherein the loading device comprises a distal seal, and wherein the method further comprises the step of inserting the sheath through said distal seal.

20. The method according to claim 18, further comprising the step of connecting the sheath with a delivery catheter.

21. The method according to claim 20, further comprising the step of advancing the embolization device through the sheath and into the delivery catheter.

22. (canceled)
